(19) United States
(12) Patent Application Publication
(10) Pub. No.: US 2013/0150811 A1
(43) Pub. Date: Jun. 13, 2013

(54) High-Flow Port Stem

(71) Applicant: Thomas J. Horgan, Gansevoort, NY (US)

(72) Inventor: Thomas J. Horgan, Gansevoort, NY (US)

(73) Assignee: AngioDynamics, Inc., Latham, NY (US)

(21) Appl. No.: 13/316,870

(22) Filed: Dec. 12, 2011

(51) Int. Cl. A61M 5/00; B23B 35/00

(52) U.S. Cl. 604/288.01; 408/1 R

(57) ABSTRACT

An implantable port with a high-flow stem for fluid access to a site within a human body is disclosed. The port has a reservoir and a needle-penetrable septum for access to the reservoir. The port also has a stem with multiple cylindrical lumens extending through the length of the stem, and each cylindrical lumen is in fluid communication with the reservoir.

### TECHNICAL FIELD

The present invention relates generally to an implantable port for fluid access to a site within a human body. More specifically, the invention relates to an implantable port with a reservoir, a needle-penetrable septum for access to the reservoir, and a stem with multiple cylindrical lumens in fluid communication with the reservoir.

### BACKGROUND OF THE INVENTION

Ports are commonly implanted in humans for fluid access to a site within the body. Ports generally include a reservoir, a needle-penetrable septum for access to the reservoir, and a stem for connecting a catheter to the port and transmitting fluid between the reservoir and a target site within the body. The outside of the stem can be shaped to accept a catheter, and a locking mechanism is typically used to secure the catheter to the stem. The distal end of the catheter can be advanced to a target site within the body. The target site can vary depending on the type of treatment being administered.

Treatments such as antibiotics, chemotherapy, pain medicine, and nutrition are commonly infused transdermally into the port reservoir, and delivered to the target site. For example, a syringe containing a treatment fluid can be connected to a hypodermic needle for infusing treatment through the port. Once the needle fully penetrates the skin and the septum, there is fluid access between the port reservoir and the syringe. Treatment fluid can then be infused into the reservoir, through a lumen in the stem, through the lumen in the catheter, and delivered to the target site. The port can also be used for aspirating fluid transdermally from the target site to an external container. For example, a syringe can be used to create negative pressure in the reservoir and the attached catheter. As a result, fluid near the tip of the catheter will flow through the catheter, through the stem, into the reservoir, through the hollow opening in the needle tip, and into the syringe. Huber style needles are commonly employed for establishing transdermal fluid access to a port reservoir.

Ports with high flow rates are desirable for numerous treatments. For example, during contrast enhanced computed tomography scans, it is desirable to inject contrast media to the target site at a high flow rate to facilitate improved imaging contrast and clarity. Alternatively, for apheresis and dialysis applications, infusion and aspiration of fluid must occur at high flow rates for proper treatment.

In a single reservoir port, the stem is typically a tubular shaped stem made of the same material as the port body. Common port body and stem materials include plastic, stainless steel or titanium. Various techniques are used to create a fluid channel or lumen through the center of the stem, often depending on the composition of the stem. For example, for a plastic stem, cylindrical and non-cylindrical lumens can easily be created using molding and manipulation techniques commonly known in the art. In one technique, a metal insert shaped to the desired dimensions of the lumen is embedded into the stem during molding. Once the molding process is complete, the metal insert is removed, creating a lumen characterized by the shape of the metal insert. For stem compositions that are harder to shape, such as metals, a small drill bit can be used to drill a cylindrical lumen longitudinally through the center of the stem. With drilling techniques, a lumen having a maximum cylindrical cross-sectional area can be formed by simply selecting a drill bit of appropriate diameter.

Dual reservoir port designs increase flexibility in treatment options available to the patient. Dual reservoir ports typically use opposing D-shaped stems to realize the largest cross-sectional area, while maintaining an overall circular shape capable of accepting a catheter lumen. The opposing D-shaped stem also maintains structural integrity while maintaining the separation of flows. Ideally, to maintain a high flow rate, the D-shaped stem would also have D-shaped lumen. For stems made of plastic, a D-shaped lumen can easily be created using cost-effective molding and manipulation techniques known in the art. However, for non-plastic materials that are difficult to mold and manipulate, such as metals, it is difficult to shape a non-cylindrical lumen. Manufacturing a non-cylindrical lumen in a small metal component can also be expensive and often cost prohibitive. For example, a process known as wire electric discharge machining can be used for forming non-cylindrical lumens in metals such as titanium. The wire electric discharge machining process requires the design and maintenance of special electrodes, which can dramatically increase manufacturing costs. Because of the cost and complexity involved with manufacturing non-cylindrical lumens in materials such as metals, the drilling technique as described above is often replicated in D-shaped stems, and a drill bit is used to create a cylindrical lumen of maximum diameter. Nonetheless, a cylindrical lumen of maximum diameter in a D-shaped stem has a limited flow rate, and the need remains for a cost effective way of increasing flow rates in a D-shaped stem to accommodate high flow treatments.

### SUMMARY

The present invention is directed to a port stem for an implantable port having fluid access to a site within a human body.

In one embodiment, an implantable port for fluid access to a site within a human body includes a reservoir, a needle-penetrable septum for access to the reservoir, and a stem. The stem has a plurality of cylindrical lumens extending longitudinally therethrough in fluid communication with the reservoir.

In another embodiment, an implantable dual reservoir port for fluid access to a site within a human body includes a first and second reservoir, a first needle-penetrable septum for access to at least one of the first and second reservoir, and a first and second stem. The first stem has a first plurality of cylindrical lumens extending longitudinally therethrough in fluid communication with the first reservoir, and the second stem has a second plurality of cylindrical lumens extending longitudinally therethrough in fluid communication with the second reservoir.

In another embodiment, a method for manufacturing a D-shaped stem for an implantable port includes forming the D-shaped stem, where the stem includes a proximal end and a distal end. Multiple lumens are drilled longitudinally through the stem, and the lumens extend from the proximal end to the distal end of the stem.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a perspective view of a prior art port and stem;

FIG. 2 is a perspective view of a prior art dual reservoir port and stem;

FIG. 3 is a perspective view of a port and stem according to an exemplary embodiment of the present invention;

FIG. 4 is a cross-sectional view of a prior art stem and a stem according to an exemplary embodiment of the present invention;

FIG. 5 is a cross-sectional view of the port according to an exemplary embodiment of the present invention;

FIG. 6 is a diagram comparing the cross-sectional area of single maximum diameter circle in a D-shaped space to the cross-sectional area of two offset maximum diameter circles in a D-shaped space; and

FIG. 7 is a flow chart of a method for manufacturing a high-flow port stem according to an exemplary embodiment of the present invention.

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein is a high-flow port stem.

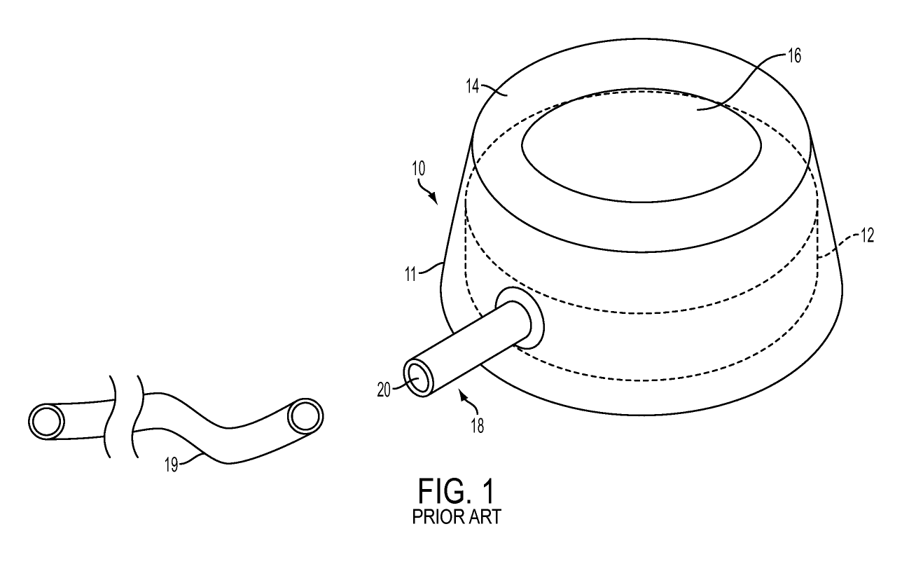

FIG. 1 shows a prior art single reservoir port 10 including a reservoir 12, a needle-penetrable septum 16, a septum retainer 14 and a stem 18. The septum 16 functions to retain fluid within the port 10, but also allows the reservoir 12 to be accessed by a needle, such as a hypodermic needle, for transdermal fluid access to the reservoir 12. The septum 16 is typically made of an elastomeric material, such as silicone or rubber, and it can be substituted for any suitable access means, such as a rubber valve. The reservoir 12 is commonly shaped as a cylinder or some other contoured shape to facilitate optimal fluid dynamics within the reservoir 12. The reservoir can be composed of the same material as the port body 11, which is typically plastic or metal, such as titanium. The stem 18 functions to provide fluid communication between the reservoir 12 and a site within the body. Prior art stems typically have a tubular shape with a single lumen 20 of constant diameter running longitudinally through the center of the stem 18. For a tubular stem composed of a hard to shape material such as metal, the lumen 20 diameter can easily be maximized by simply selecting a drill bit of appropriate diameter to create the lumen 20. The maximum cylindrical cross-sectional area of the lumen is determined with consideration given to maintaining of a level of structural integrity in the stem. The outside of the stem 18 may have one or more barbs or other shapes for accepting and securing a catheter 19. The stem 18 can also extend out from different locations on the reservoir 12, for example tangent from a side of the reservoir, or tangent to a contour located at the bottom of the reservoir.

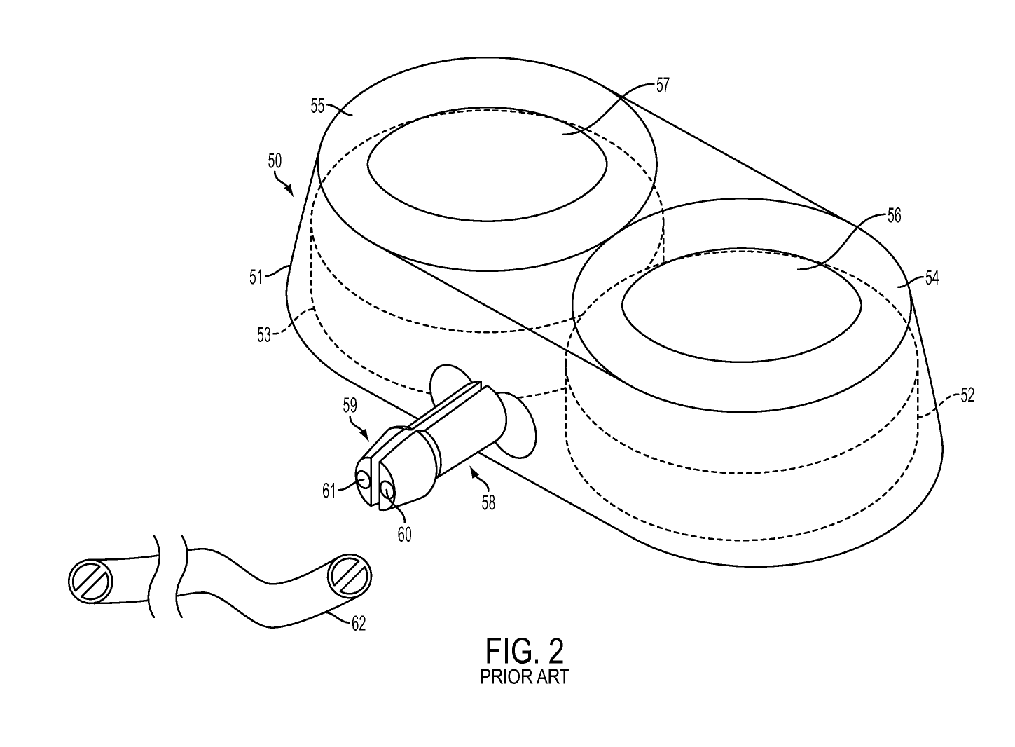

FIG. 2 shows a prior art dual reservoir port 50. In dual reservoir port designs, two separate reservoirs 52, 53 are commonly built into a unitary port body 51 such that each reservoir maintains a separation of fluid flow. A first septum 56 can be secured to the port body 51 over the first reservoir 52 by a first retaining ring 54, while a second septum 57 can be secured to the port body 51 over the second reservoir 53 by a second retaining ring 55. The retaining rings and septums could also be unitary components. A cylindrical lumen 60 is drilled into the first stem 58 for providing fluid access to the first reservoir 52, and another cylindrical lumen 61 is drilled into the second stem 59 for providing fluid access to the second reservoir 53. The first stem 58 and second stem 59 are each referred to as D-shaped, since the outline of their cross-section is substantially shaped like the letter D. Relative to each other, the first stem 58 and the second stem 59 are in an opposing D-shaped configuration, and a circular catheter with opposing D-shaped lumens 62 can be accepted onto the stems, while maintaining separation of fluids.

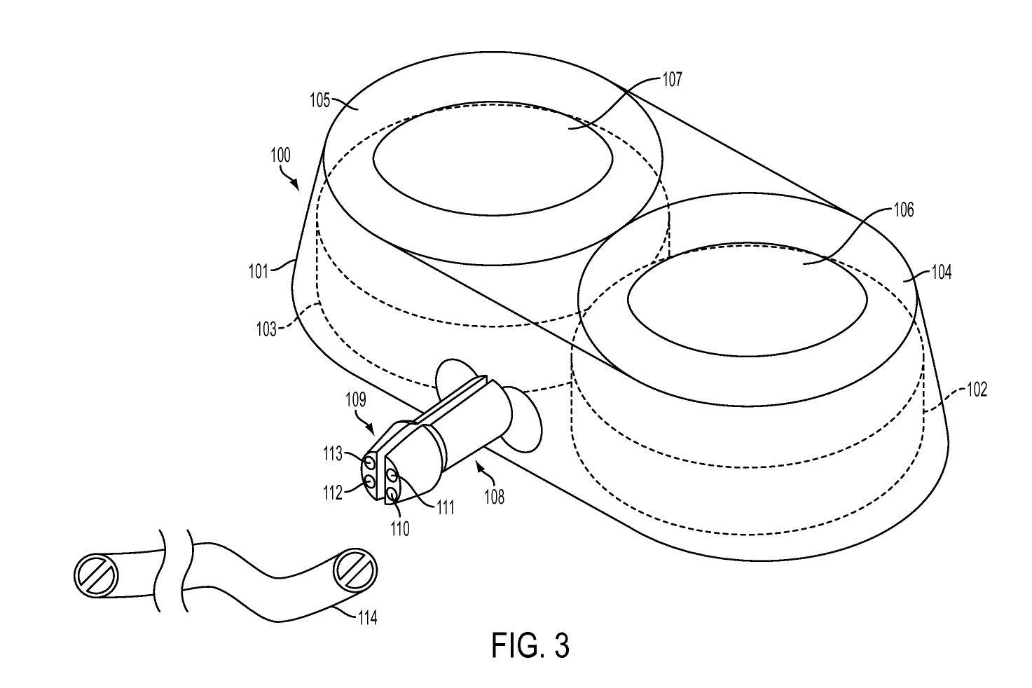

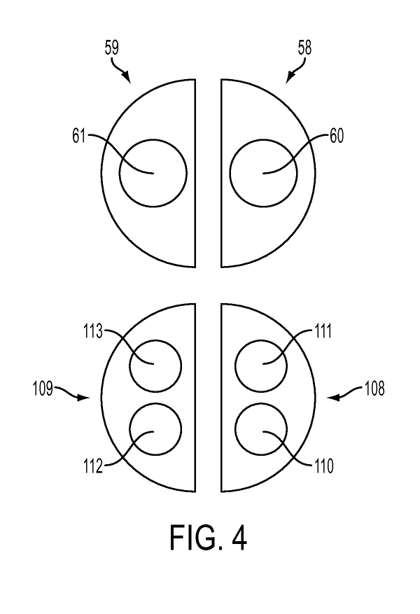

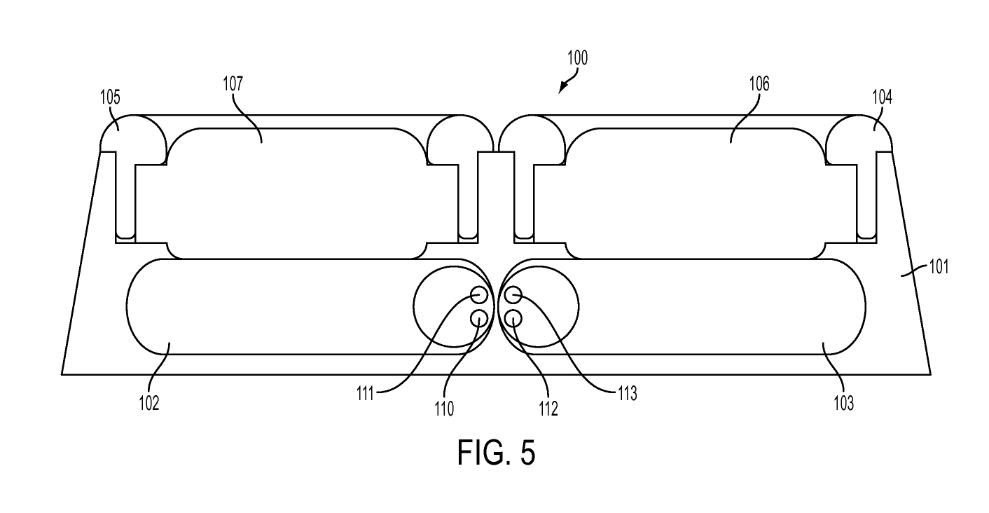

FIG. 3 shows a dual reservoir port according to an exemplary embodiment of the present invention. The first septum 106 is secured to the port body 101 over the first reservoir 102 by the first retaining ring 104, and the second septum 107 is secured to the port body 101 over the second reservoir 103 by the second retaining ring 105. The first stem 108 provides fluid access to the first reservoir 102 while the second stem 109 provides fluid access to the second reservoir 103. The first and second stems 108, 109 are each D-shaped and in an opposing D-shaped configuration, so that a catheter with opposing D-shaped lumens 114 can be accepted onto the stems, while maintaining a separation of fluids. Two cylindrical lumens 110, 111 are drilled longitudinally into the first stem 108. As shown in FIGS. 3 and 4, the lumens 110, 111 are offset from each other so that they do not overlap, while also spaced from the outer wall of the stem to maintain adequate structural integrity. As shown in FIG. 5, these lumens 110, 111 provide fluid access to the first reservoir 102. Similarly, two cylindrical lumens 112, 113 are drilled longitudinally into the second stem 109 for providing fluid access to the second reservoir 103. By utilizing two smaller offset cylindrical lumens as shown in FIGS. 3, a greater total cross-sectional area is achieved versus the single maximum diameter cylindrical lumen as shown in prior art FIG. 2. A barb or other shape can be configured on the outside of the stem to secure the catheter 114 to each stem. The stem may also be configured to accept a port catheter lock.

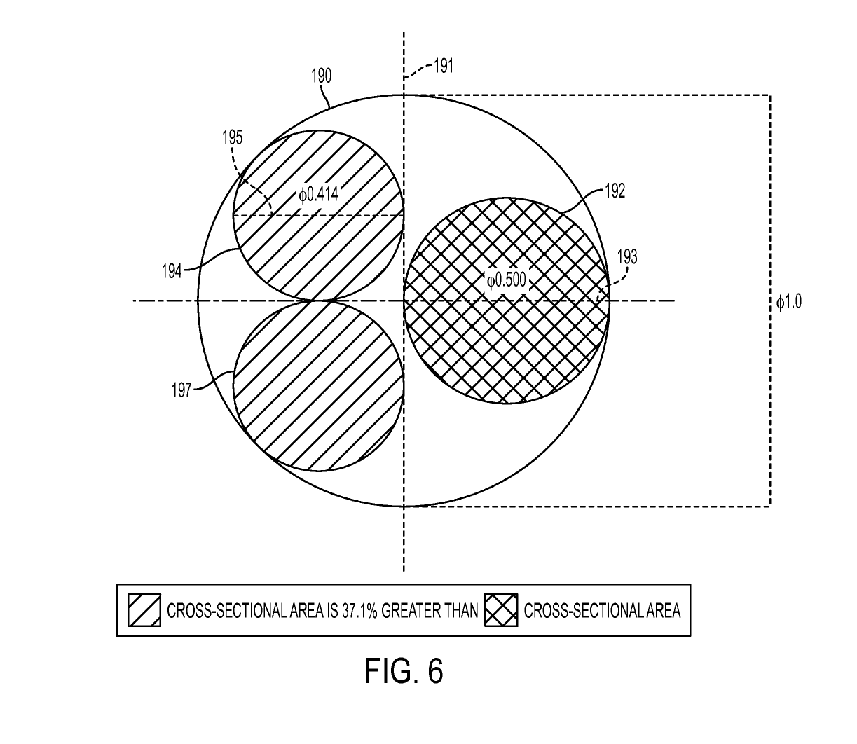

FIG. 6 shows a diagram comparing the cross-sectional area of a maximum diameter circle to the cross-sectional area of two smaller offset circles. A circle 190 of diameter 1.0 is bisected by a line 191 to form two equivalent and opposing D-shaped areas. On the right, a single maximum diameter circle 192 has a diameter 193 of 0.500, while on the left side, two smaller offset circles 194, 197 have a diameter 195 of approximately 0.414. As illustrated by the diagram, the cross-sectional area of the two smaller offset circles is approximately 37.1% greater than the cross-sectional area of the maximum diameter circle. Similarly, in a D-shaped stem, two offset cylindrical lumens of a smaller diameter will achieve a higher total cross-sectional area than a single cylindrical lumen of maximum diameter. As a result of the higher total cross sectional area, the present embodiment yields a higher flow rate in comparison to the prior art design shown in FIG. 2.

A flow simulator was used to compare the flow rate of a D-shaped stem having a single maximum diameter lumen with the flow rate of an equivalent D-shaped stem having two smaller offset lumens. The diameter of the single maximum lumen was 0.038 inches, and the diameter of each offset lumen in the two lumen design was 0.032 inches. For equivalent pressure, fluid viscosity and stem length, the two lumen design resulted in a flow rate improvement factor of approximately 3.26 times the flow rate of the single maximum lumen. An additional advantage of the two lumen design is a lower pressure drop across the stem. A simulation using 11.8 cP contrast media at a flow rate of 5 ml/s resulted in a pressure drop of 304 mmHg measured across the stem for the single maximum lumen design, and a pressure drop of 167 mmHg measured across the stem for the two offset lumen design, representing an approximately 45% improvement. Lower pressure levels provide a clinical advantage in both blood conveyance and contrast media injections by maximizing injector performance, minimizing the possibility of patient injury, lowering pressure levels in the port assembly and lessening strain on the catheter shaft.

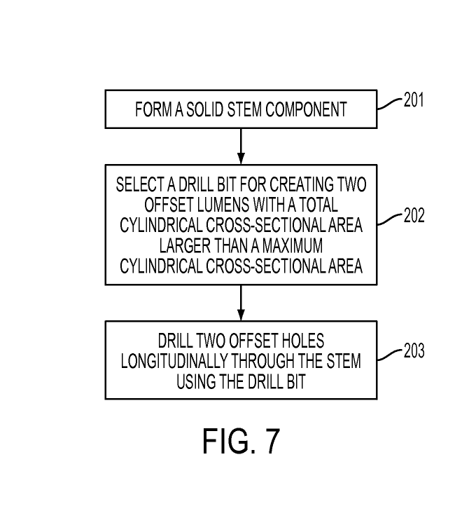

FIG. 7 shows a flow chart for a method of manufacturing a port stem 200. The port body, septum, septum retainer and stem can be manufactured as separate components, then assembled to make the port. As mentioned above, it is difficult and often cost-prohibitive to shape a non-cylindrical lumen in a stem made from materials such as metal compositions. For example, a common method for forming a D-shaped lumen involves using wire electric discharge machining, which requires the design and maintenance of special electrodes, and can more than double manufacturing costs. However, a high-flow port stem can be manufactured in a cost-effective manner by first forming a solid stem component 201. Metal components of this size and shape can be manufactured on Swiss-type turning machines. A drill bit of appropriate diameter can be selected 202, and two holes can be drilled longitudinally through the stem, extending from the proximal end to the distal end of the stem 203. The drilling can be accomplished by selecting a small drill bit of appropriate diameter to create two cylindrical lumens with a total cross-sectional area greater than the maximum cylindrical cross-sectional area, giving consideration to maintaining the structural integrity of the stem. The stem component can then be finished in a tumbling operation and then passivated. This method of manufacture can efficiently and cost-effectively be implemented in stems composed of hard to shape metals, such as titanium. Additionally, since metal has a higher structural integrity than plastic, metal stems can be manufactured with smaller margins of wall thickness for maintaining structural integrity versus their plastic counterparts. Thus, for smaller stems, it is possible for a metal D-shaped stem with two offset cylindrical lumens to achieve a higher total cross-sectional area than a plastic D-shaped stem of equivalent outer dimension having a D-shaped lumen. That is, since a plastic stem requires a higher margin of wall thickness for maintaining structural integrity, a D-shaped lumen in a plastic stem would have to be downsized to accommodate the required wall thickness, resulting in a comparatively larger total cross-sectional area for the equivalent metal D-shaped stem having two offset cylindrical lumens.

1. An implantable port for fluid access to a site within a human body, comprising:
a reservoir;
a needle-penetrable septum for access to the reservoir; and
a stem having a plurality of cylindrical lumens extending longitudinally therethrough in fluid communication with the reservoir.

2. The port of claim 1,
wherein the plurality of cylindrical lumens have a total cross-sectional area, and
wherein the total cross-sectional area is greater than a maximum cylindrical cross-sectional area.

3. The port of claim 1, wherein a cross sectional outline of the stem is D-shaped.

4. The port of claim 1, wherein the plural of cylindrical lumens is two.

5. The port of claim 1, wherein the stem comprises a metal composition.

6. An imp table dual reservoir port for fluid access to a site within a human body, comprising:
a first and second reservoir;
a first needle-penetrable septum for access to at least one of the first and second reservoir;
a first stem having a first plurality of cylindrical lumens extending longitudinally therethrough in fluid communication with the first reservoir; and
a second stem having a second plurality of cylindrical lumens extending longitudinally therethrough in fluid communication with the second reservoir.

7. The dual reservoir port of claim 6, wherein the first and second stems form an opposing D-shaped cross-sectional outline.

8. The dual reservoir port of claim 6,
wherein the plurality of cylindrical lumens have a total cross-sectional area, and
wherein the total cross-sectional area is greater than a maximum cylindrical cross-sectional area.

9. The dual reservoir port of claim 6, wherein the plurality of cylindrical lumens is two.

10. The port of claim 6, wherein the stem comprises a metal composition.

11-14. (canceled)
